(19) United States
(12) Patent Application Publication
(10) Pub. No.: US 2014/0066719 A1
(43) Pub. Date: Mar. 6, 2014

(54) SURGICAL RETRACTOR AND METHOD OF USE

(71) Applicant: Paula A. Nichter, Memphis, TN (US)

(72) Inventor: Paula A. Nichter, Memphis, TN (US)

(73) Assignee: WARSAW ORTHOPEDIC, INC., Warsaw, IN (US)

(21) Appl. No.: 13/602,817

(22) Filed: Sep. 4, 2012

(51) Int. Cl. A61B 1/32

(52) U.S. Cl. 600/215

(57) ABSTRACT

A surgical retractor comprises a first member extending between a first end and a second end including at least one first fixation element. A second member includes a blade and a biasing member connected with the blade. The biasing member includes at least one second fixation element that is selectively engageable with the at least one first fixation element to releasably dispose the blade in a selected orientation relative to the first end. Systems and methods of use are disclosed.

### TECHNICAL FIELD

The present disclosure generally relates to medical devices for the treatment of musculoskeletal disorders, and more particularly to a surgical system and method for accessing a surgical site to facilitate treatment.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes fusion, fixation, discectomy, laminectomy and implantable prosthetics. Surgical retractors may be employed during a surgical treatment to provide access and visualization of a surgical site. Such retractors space apart and support tissue and/or other anatomical structures to expose anatomical structures adjacent the surgical site and/or provide a surgical pathway to the surgical site. This disclosure describes an improvement over these prior art technologies.

### SUMMARY

Accordingly, a surgical system and method are provided for accessing a surgical site, which may include, for example, a portion of a spine to facilitate treatment thereof. It is contemplated that the surgical system and method may be employed for exposing and providing a surgical pathway to a surgical site.

In one embodiment, in accordance with the principles of the present disclosure, a surgical retractor is provided. The surgical retractor comprises a first member extending between a first end and a second end including at least one first fixation element. A second member includes a blade and a biasing member connected with the blade. The biasing member includes at least one second fixation element that is selectively engageable with the at least one first fixation element to releasably dispose the blade in a selected orientation relative to the first end.

### BRIEF DESCRIPTION OF THE DRAWINGS

The present disclosure will become more readily apparent from the specific description accompanied by the following drawings, in which:

FIG. 1 is a perspective view, in part phantom, of one embodiment of components of a system in accordance with the principles of the present disclosure;

FIG. 2 is a perspective view of a component of the system shown in FIG. 1;

FIG. 3 is a perspective view of components of the system shown in FIG. 1;

FIG. 4 is a break away perspective view of components of the system shown in FIG. 1;

FIG. 5 is a break away perspective view of components of the system shown in FIG. 1; and

FIG. 6 is a side cutaway view of the system shown in FIG. 4.

Like reference numerals indicate similar parts throughout the figures.

### DETAILED DESCRIPTION

The exemplary embodiments of the surgical system and related methods of use disclosed are discussed in terms of medical devices for the treatment of musculoskeletal disorders and more particularly, in terms of a surgical system for accessing a spine to facilitate treatment thereof and a method for treating a spine. The surgical system and methods presently disclosed provide facile assembly of a retractor system for connection of the mating parts. One or all of the system components may be reusable or disposable. The surgical system may be configured as a kit with multiple sized and configured components.

In one embodiment, the system includes a surgical retractor having at least one adjustable length retractor blade that includes locking teeth according to the requirements of a particular application. It is envisioned that the adjustable blades avoid tissue creep about the blade and into a surgical site. In one embodiment, the retractor comprises blades that prevent tissue progression in the workspace. It is contemplated that the blades avoid undesired tissue creep into the workspace and/or tissue damage such as, for example, of the lungs, bowels or other anatomy. In one embodiment, the system includes a retractor having adjustable length retractor blades that allow custom fit of the blades to a particular application or patient anatomy.

In one embodiment, the system includes a surgical retractor having at least one adjustable length retractor blade, which includes a blade top and a blade bottom. In one embodiment, the blade top includes a connection to a retractor frame and ratchet teeth disposed along its length. In one embodiment, the blade bottom includes a leaf spring lock with ratchet teeth similar to the teeth of the blade top. The blade bottom has a geometry to grab tissue. It is contemplated that the leaf spring lock may be manufactured separately and attached to the blade bottom. In one embodiment, the leaf spring lock includes grips with fingers so that the leaf spring lock can be pulled back, or disengaged, from the blade top, such that the leaf spring lock is released at a selected length of the blade bottom relative to the blade top. In one embodiment, the surgical retractor comprises a sliding adjustable length blade and includes a blade top that does not include teeth adjacent a middle section thereof. In one embodiment, the leaf spring is non-locking.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the disclosed surgical system and methods may be alternatively employed in a surgical treatment with a patient in a prone or supine position, and/or employ various surgical approaches to the spine, including anterior, posterior, posterior mid-line, lateral, postero-lateral, and/or antero-lateral approaches, and in other body regions. The present disclosure may also be alternatively employed with procedures for treating the lumbar, cervical, thoracic and pelvic regions of a spinal column. The system and methods of the present disclosure may also be used on animals, bone models and other non-living substrates, such as, for example, in training, testing and demonstration.

The present disclosure may be understood more readily by reference to the following detailed description taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

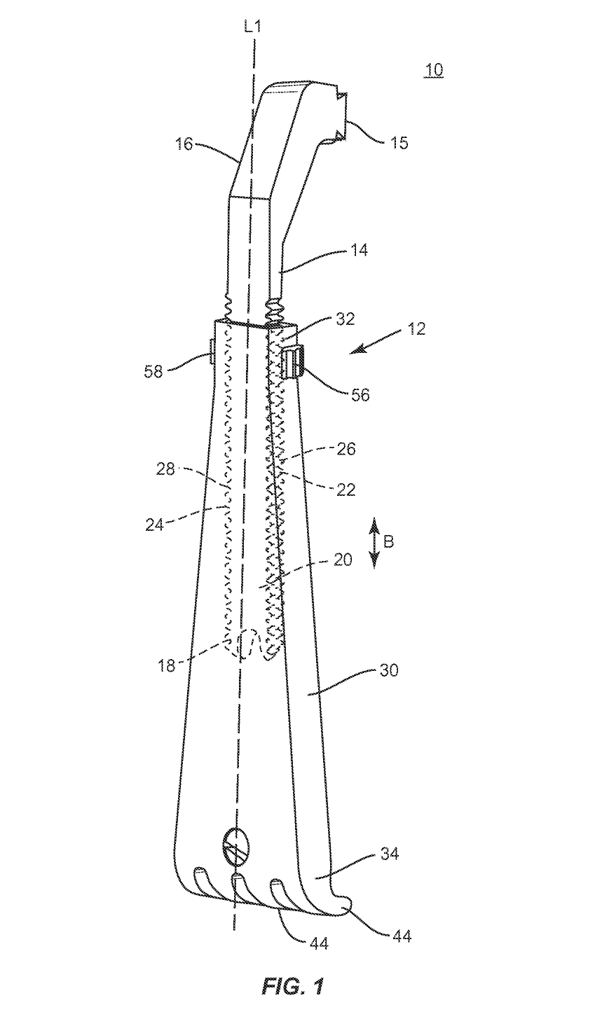

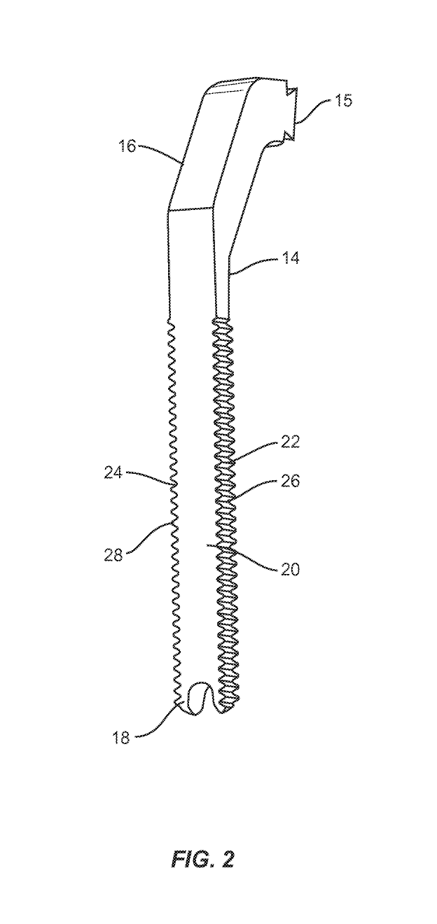

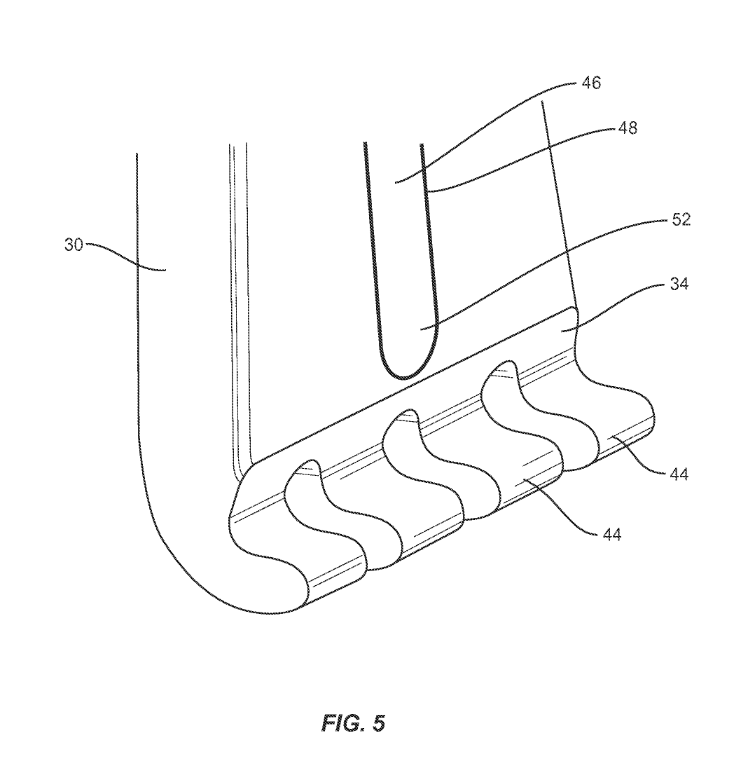

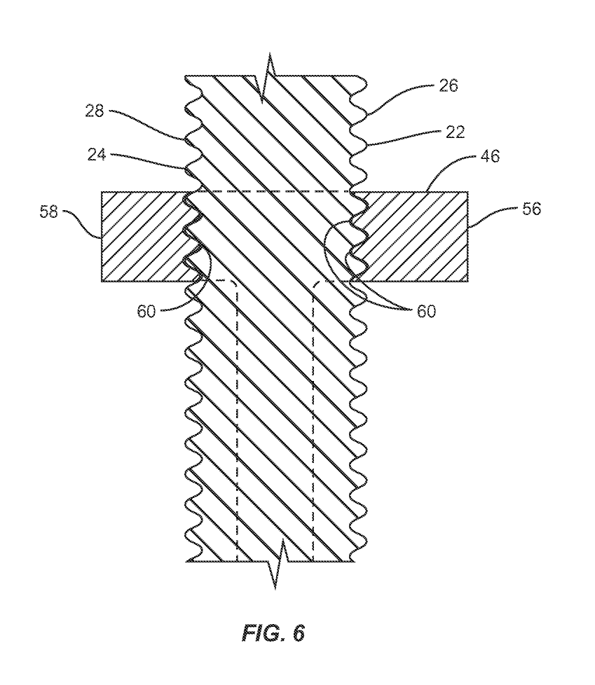

The following discussion includes a description of a surgical system and related methods of employing the surgical system in accordance with the principles of the present disclosure. Alternate embodiments are also disclosed. Reference will now be made in detail to the exemplary embodiments of the present disclosure, which are illustrated in the accompanying figures. Turning now to FIGS. 1-6, there is illustrated components of a surgical system 10 including a retractor for accessing a spine to facilitate treatment thereof in accordance with the principles of the present disclosure.

The components of surgical system 10 can be fabricated from biologically acceptable materials suitable for medical applications, including metals, synthetic polymers, ceramics and/or their composites, depending on the particular application and/or preference of a medical practitioner. For example, the components of surgical system 10, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, composites of PEEK and calcium based ceramics, and composites of PEEK with resorbable polymers. Various components of surgical system 10 may have material composites, including the above materials, to achieve various desired characteristics such as strength, rigidity, elasticity, compliance, biomechanical performance, durability and radiolucency or imaging preference. The components of surgical system 10, individually or collectively, may also be fabricated from a heterogeneous material such as a combination of two or more of the above-described materials. The components of surgical system 10 may be monolithically formed, integrally connected or include fastening elements and/or instruments, as described herein.

Surgical system 10, is employed, for example, with minimally invasive, mini-open and/or open procedures for supporting tissue and/or anatomical structures to expose tissue and/or anatomical structures to create a surgical pathway and provide access to a surgical site, which includes, for example, a spine to facilitate treatment.

Surgical system 10 includes a retractor assembly 12 including a first member, such as, for example, a rack 14 configured for attachment to a base 15. Rack 14 defines a longitudinal axis L1. Rack 14 extends between a first end 16 and a second end 18. Base 15 is configured for connection with a frame (not shown). The frame allows for proper positioning and docking of retractor assembly 12 with an anatomy in alignment with the surgical site. It is contemplated that base 15 may be monolithically formed, integrally connected and/or include fastening elements that attach base 15 with the frame. In one embodiment, retractor assembly 12 may be employed with a free hand surgical technique such that base 15 is not connected to a frame.

End 16 is disposed at an angular orientation relative to axis L1. It is contemplated that end 16 may be oriented in alternate configurations, such as, for example, perpendicular, parallel, co-axial, angularly offset, offset and/or staggered relative to longitudinal axis L1.

End 18 defines a fixation surface 20 for engagement and disengagement with a fixation surface of a biasing member, described herein. Fixation surface 20 defines a first face 22 and a second face 24. Face 22 includes at least one fixation element, such as, for example, a plurality of teeth 26 and face 24 includes at least one fixation element, such as, for example, a plurality of teeth 28. It is contemplated that face 22 and/or face 24 may include one or a plurality of fixation elements. It is further contemplated that a fixation element may alternatively comprise a nail, key, keyway, nub, friction fit, detent, post and/or releasable locking structure. It is further contemplated that face 22 and face 24 include various surface configurations, such as, for example, smooth, rough, mesh, porous, semi-porous, dimpled and/or textured according to the requirements of a particular application.

A second member, such as, for example, a tissue engaging blade 30 extends between a first end 32 and a second end 34. Blade 30 extends in a tapered configuration such that blade 30 has an increasing dimension, such as, for example, width, thickness, diameter and/or height, from end 32 to end 34. As such, end 34 has a greater width than end 32 for engaging tissue.

Blade 30 includes an inner surface 31 and an outer surface 33. Inner surface 31 defines a longitudinal cavity 36. Cavity 36 extends from end 32 to end 34. Cavity 36 is configured for disposal of rack 14. It is contemplated that the cross-section of cavity 36 may have various configurations, for example, round, oval, polygonal, rectangular, irregular, tapered, staggered, uniform and non-uniform.

Outer surface 33 defines a longitudinal cavity 38. Cavity 38 includes a longitudinal portion 40 and a transverse portion 42 that communicates with portion 40. Portion 40 extends from end 32 to end 34. Portion 42 extends circumferentially about a portion of end 32 and connects with portion 40 in a configuration for disposal of the biasing member, described herein. It is contemplated that the surfaces defining cavity 36 and/or cavity 38 may include various surface configurations, such as, for example, smooth, rough, mesh, porous, semi-porous, dimpled and/or textured according to the requirements of a particular application. It is further contemplated that blade 30 may be tubular, cylindrical, elliptical, polygonal, circular or non-circular, irregular, tapered, staggered, uniform and non-uniform in cross-section.

Blade 30 includes at least one foot, such as, for example, feet 44 disposed at end 34. Feet 44 are disposed in a transverse orientation relative to axis L1. It is envisioned that feet 44 may be oriented in alternate configurations, such as, for example, perpendicular, parallel, co-axial, angularly offset, offset and/or staggered relative to axis L1. Feet 44 are configured to engage tissue. In one embodiment, feet 44 are spaced apart along end 34.

A biasing member, such as, for example, a leaf spring 46 is configured for disposal with portions 40, 42. Spring 46 has a cruciate configuration and includes a longitudinal portion 48 configured for disposal with portion 40 and fixed with a distal end, such as, for example, end 34 of blade 30 such that a proximal portion of spring 46 can rotate relative to end 34. Portion 48 is fixed with end 34 via a screw. It is envisioned that portion 40 may alternatively be fixed with end 34 via integral connection or monolithic formation with blade 30, clip, nail, rivet and/or adhesive. It is further envisioned that the biasing member may alternatively include a spring, coil spring, hinge and/or an elastic member such as a tether. In one embodiment, blade 30 includes a rigid member that is disposed with cavity 38 and rotatable and/or pivotable relative to end 34.

Portion 48 extends between a first end 50 and a second end 52 along axis L1. End 50 includes a transverse portion 54 disposed with a proximal end, such as, for example, end 32 of blade 30 and configured for disposal in portion 42 of cavity 38. Portion 54 includes a first arm 56 and a second arm 58. Arms 56, 58 extend outwardly in an opposite direction from each other from end 50 transverse to axis L1. Arms 56, 58 are configured for disposal in transverse portion 42. It is contemplated that arm 56 and/or arm 58 may be oriented in alternate configurations, such as, for example, perpendicular, parallel, co-axial, angularly offset, offset and/or staggered relative to longitudinal axis L1.

Transverse portion 54 defines a fixation surface configured for movable engagement and disengagement with surface 20 of rack 14 via pivoting motion of spring 46. The fixation surface of spring 46 includes surfaces of arms 56, 58 that are disposed in opposing relation such that the surfaces are oriented to face each other and the fixation surface of rack 14. The fixation surfaces of each of arms 56, 58 include at least one fixation element, such as, for example, a plurality of teeth 60. It is contemplated that fixation surfaces of each of arms 56, 58 may include one or a plurality of fixation elements. It is further contemplated that a fixation element may alternatively comprise a nail, key, keyway, nub, friction fit, detent, post and/or releasable locking structure. It is further contemplated that fixation surfaces of each of arms 56, 58 may include various surface configurations, such as, for example, smooth, rough, mesh, porous, semi-porous, dimpled and/or textured according to the requirements of a particular application. Teeth 60 of each of arms 56, 58 are configured for engagement with teeth 26, 28, respectively, of rack 14.

Each of arms 56, 58 include an outer surface 62 that defines a gripping surface, such as, for example, knurled finger grip 64. It is contemplated that outer surface 62 includes various surface configurations, such as, for example, smooth, rough, mesh, porous, semi-porous, dimpled and/or textured according to the requirements of a particular application.

The resilient configuration of spring 46 facilitates rotation of transverse portion 54 relative to second end 34, via manual manipulation thereof by a practitioner, such that teeth 60 are selectively engageable and disengageable with teeth 26, 28 for pivotable movement of spring 46 relative to rack 14 and blade 30. Spring 46 resiliently biases teeth 60 into removable fixation with teeth 26, 28. As such, blade 30 is selective adjustable between a first configuration such that blade 30 is adjustably slidable relative to rack 14 and a second configuration such that blade 30 is disposed in a selected orientation relative to first end 16 of rack 14, and/or the frame connected to rack 14, in a releasably lockable position. It is contemplated that such selective adjustment of blade 30 may include a range of displacement of blade 30 relative to rack 14, and/or may include increments of 1 centimeter.

In one embodiment, blade 30 is selectively adjustable in a range of extension from rack 14, for example, from a first orientation, such as, for example, minimum extended position such that end 34 is disposed in close proximity to end 18 and a second orientation, such as, for example, maximum extended position such that end 34 is spaced apart from end 18 and end 18 is disposed in a close proximity to end 32. It is envisioned that blade 30 may be disposed in the second configuration in a releasably locked orientation in a plurality of positions in the range of extension.

In assembly, operation and use, surgical system 10, similar to that described with regard to FIGS. 1-6, is employed, for example, with a minimally invasive surgical procedure for spinal and neurosurgical applications with a patient. For example, during spine surgery, a surgeon will make an incision in the skin of a patient's back over vertebrae to be treated. One or more dilators may be employed to gradually separate the muscles and create a portal through which the surgery may be performed.

Retractor assembly 12 is positioned adjacent the surgical site over the small incision. Retractor assembly 12 is passed through the incision to create a passageway or portal to the surgical site. Blade 30 is disposed such that teeth 60 are releasably engaged with teeth 26, 28. Second end 18 is disposed adjacent second end 34 of blade 30, for example, in the minimum extended position described above.

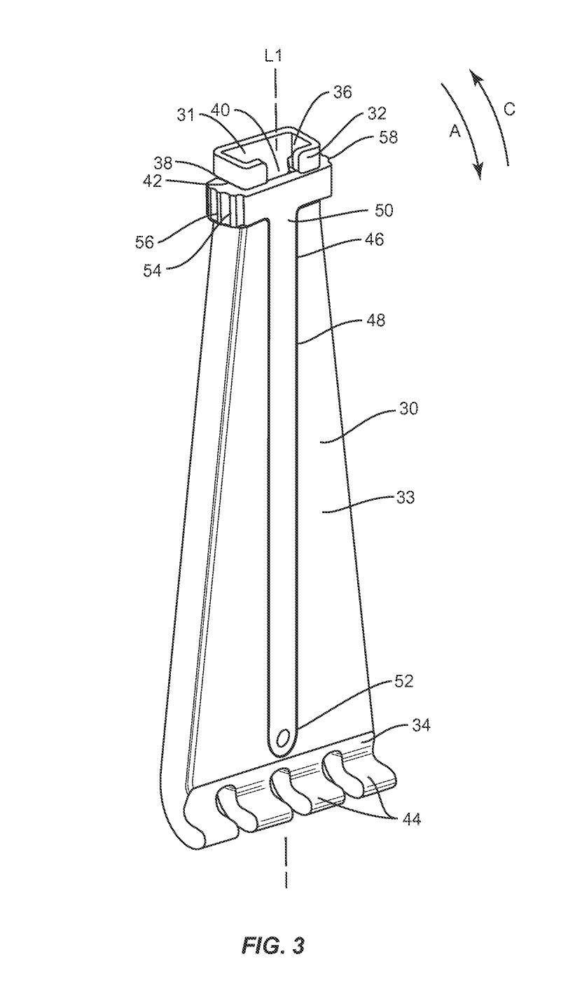

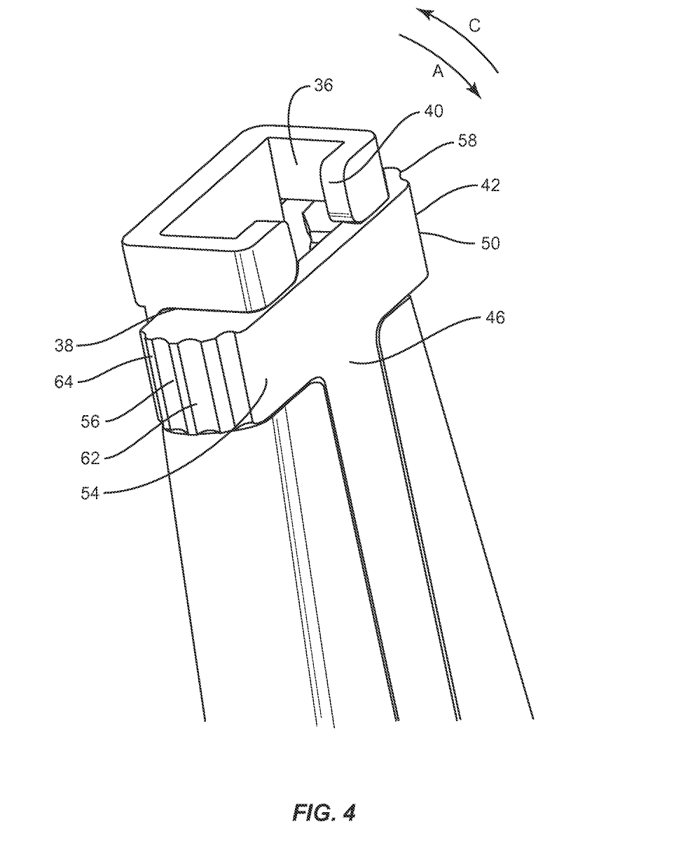

To extend or retract blade 30 relative to rack 14/frame to one or a plurality of positions in the range of extension, blade 30 is selectively adjusted corresponding to the first configuration described above. The practitioner manipulates arms 56, 58, in a direction shown by arrow A in FIGS. 3 and 4. The resilient configuration of spring 46 facilitates rotation of portion 54 such that teeth 60 to disengage from teeth 26, 28. Blade 30 is freely slidable relative to rack 14/frame for axial translation along longitudinal axis L1, in the direction shown by arrows B in FIG. 1.

Upon selective extension or contraction of blade 30 within the range of extension relative to rack 14/frame, arms 56, 58 are released by the practitioner from the first configuration and portion 54 is biased into portion 42, in the direction shown by arrow C in FIGS. 3 and 4, such that blade 30 is disposed in a releasably locked orientation of the second configuration. Teeth 60 engage teeth 26, 28 for releasable fixation of blade 30 in the selected orientation.

Blade 30 and/or feet 44 engage tissue to separate tissue adjacent the surgical site and/or prevent tissue from entering the passageway or portal at the surgical site and/or prevent tissue creep at the surgical site. In one embodiment, blade 30 can be locked in one or a plurality of orientations and/or a selected distance or dimension relative to rack 14, as described.

Upon completion of the procedure, retractor assembly 12 is removed from the surgical site. In one embodiment, blade 30 is selectively extended or contracted, as described above, and retractor assembly 12 is removed from the surgical site.

It is envisioned that the use of microsurgical and image guided technologies may be employed to access, view and repair spinal deterioration or damage, with the aid of retractor assembly 12. It is contemplated that a surgical procedure may employ other instruments that can be mounted with retractor assembly 12, such as, for example, nerve root retractors, tissue retractors, forceps, cutter, drills, scrapers, reamers, separators, rongeurs, taps, cauterization instruments, irrigation and/or aspiration instruments, illumination instruments and/or inserter instruments.

Retractor assembly 12 may be employed for performing spinal surgeries, such as, for example, discectomy, laminectomy, fusion, laminotomy, laminectomy, nerve root retraction, foramenotomy, facetectomy, decompression, spinal nucleus or disc replacement and procedures using bone graft and implantable prosthetics including plates, rods, and bone engaging fasteners.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

1. A surgical retractor comprising:
a first member extending between a first end and a second end including at least one first fixation element; and
a second member including a blade and a biasing member connected with the blade, the biasing member including at least one second fixation element that is fixed relative to a body of the biasing member, the at least one second fixation element being selectively engageable with the at least one first fixation element to releasably dispose the blade in a selected orientation relative to the first end.

2. The surgical retractor as recited in claim 1, wherein the first member defines a longitudinal axis and the first end is disposed at an angular orientation relative to the longitudinal axis.

3. The surgical retractor as recited in claim 1, wherein the at least one first fixation element includes a plurality of teeth extending along the second end.

4. The surgical retractor as recited in claim 1, wherein the second end includes a rack and the at least one first fixation element includes a plurality of teeth disposed along the rack.

5. The surgical retractor as recited in claim 1, wherein the second end includes a first face and a second face spaced apart from the first face, the at least one first fixation element including a plurality of teeth disposed along the first face and a plurality of teeth disposed along the second face.

6. The surgical retractor as recited in claim 1, wherein the blade defines a longitudinal cavity configured for disposal of the biasing member.

7. The surgical retractor as recited in claim 1, wherein the biasing member includes a longitudinal portion fixed with a distal end of the blade and a transverse portion disposed with a proximal end of the blade.

8. The surgical retractor as recited in claim 1, wherein the biasing member includes a cruciate configuration.

9. The surgical retractor as recited in claim 1, wherein the biasing member rotates relative to distal end of blade.

10. The surgical retractor as recited in claim 1, wherein the biasing member includes an outer surface that defines a gripping surface.

11. The surgical retractor as recited in claim 1, wherein the blade extends from a first end to a second end having a tapered configuration.

12. The surgical retractor as recited in claim 1, wherein the biasing member includes a first face and a second face spaced apart from the first face, the at least one second fixation element including a plurality of teeth disposed along the first face and a plurality of teeth disposed along the second face.

13. The surgical retractor as recited in claim 1, wherein the blade includes at least one foot extending therefrom.

14. The surgical retractor as recited in claim 1, wherein the blade includes a plurality of spaced apart feet configured to engage tissue.

15. The surgical retractor as recited in claim 1, wherein the biasing member includes a leaf spring.

16. The surgical retractor as recited in claim 1, wherein the biasing member is rotatable relative to a distal end of the blade such that the fixation elements are selectively engageable to dispose the members between a first configuration such that the blade is adjustably slidable relative to the first member and a second configuration such that the blade is disposed in the selected orientation relative to the first end of the first member.

17. A surgical retractor comprising:
a first member defining a longitudinal axis and extending between a first end and a second end including at least one first fixation element; and
a second member including a tissue engaging blade extending between a first end and a second end and defining a longitudinal cavity, and a biasing member configured for disposal with the longitudinal cavity, the biasing member extending between a first end including at least one second fixation element and a second end fixed with the second end of the blade, the at least one second fixation element being fixed relative to a body of the biasing member,
wherein the biasing member is rotatable relative to the second end of the blade such that the fixation elements are selectively engageable to dispose the members between a first configuration such that the blade is adjustably slidable relative to the first member and a second configuration such that the blade is disposed in a selected orientation relative to the first end of the first member.

18. The surgical retractor as recited in claim 17, wherein the longitudinal cavity includes a transverse portion and the first end of the biasing member includes a first arm and a second arm, the arms extending outwardly from the first end of the biasing member and being configured for disposal with the transverse portion.

19. The surgical retractor as recited in claim 18, wherein the at least one second fixation element includes a plurality of teeth and each of the arms includes an inner surface and an outer surface, the inner surfaces each including a portion of the teeth and the outer surfaces each including a gripping surface.

20. A surgical retractor comprising:
a rack defining a longitudinal axis and extending between a first end and a second end that defines a first face including a first plurality of teeth and a second face including a second plurality of teeth;
a tissue engaging blade extending between a first end and a second end and defining a first longitudinal cavity configured for disposal of the rack and a second cavity, the second cavity including a longitudinal portion and a transverse portion; and
a biasing member extending between a first end and a second end, the biasing member including a longitudinal portion configured for disposal with the longitudinal portion of the second cavity and a transverse portion of the second cavity, the longitudinal portion being fixed with the second end of the blade and the transverse portion of the biasing member including teeth configured for engagement with the teeth of the rack, the teeth of the biasing member being fixed relative to a body of the biasing member from which the teeth of the biasing member extend,
wherein the biasing member is rotatable relative to the second end of the blade such that the teeth of the biasing member are selectively engageable with the teeth of the rack to dispose the second end of the blade relative to the first end of the rack between a first configuration such that the blade is adjustably slidable relative to the rack and a second configuration such that the blade is disposed in a selected orientation relative to the first end of the rack.
